(19) United States
(12) Patent Application Publication
(10) Pub. No.: US 2025/0152604 A1
(43) Pub. Date: May 15, 2025

(54) CONTRACEPTIVE METHODS WITH SCHEDULED BLEEDING EFFECTS

(71) Applicant: ESTETRA SRL, Liège (BE)

(72) Inventors: Jean-Michel FOIDART, Liège (BE); Maud JOST, Liège (BE)

(73) Assignee: ESTETRA SRL, Liège (BE)

(21) Appl. No.: 18/837,118

(22) PCT Filed: Feb. 9, 2023
(86) PCT No.: PCT/IB2023/051177

§ 371 Date: Aug. 8, 2024

Related U.S. Application Data
(60) Provisional application No. 63/308,910, filed on Feb. 10, 2022.

(51) Int. Cl. A61K 31/567; A61K 31/585; A61P 15/18

(52) U.S. Cl. CPC A61K 31/567; A61K 31/585; A61P 15/18

(57) ABSTRACT

Described are methods of providing contraception with increased likelihood of scheduled bleeding in a woman having a BMI<30.0 kg/m² and methods of providing contraception with reduced likelihood of scheduled bleeding in a woman having a BMI≥30.0 kg/m². The methods comprise orally administering a therapeutically effective amount of an estetrol component at a daily dose of from 14 mg to 16 mg, based on the estetrol moiety, and drospirenone at a daily dose of from 2.5 mg to 3.5 mg. Also provided are compositions comprising an estetrol component and drospirenone for use in such methods.

### CROSS-REFERENCE TO RELATED APPLICATIONS

This application claims priority to U.S. provisional application 63/308,910, filed Feb. 10, 2022, the entire contents of which are incorporated herein by reference.

### FIELD

Described are contraceptive methods with scheduled bleeding and contraceptive methods with different likelihood of scheduled bleeding in women having different BMI levels, as well as compositions comprising an estetrol component and drospirenone for use in such methods.

### BACKGROUND

The following discussion is provided to aid the reader in understanding the disclosure and is not admitted to describe or constitute prior art thereto.

Combined Oral Contraceptives (COCs) comprising a progestin component and an estrogen component are commonly used for contraception worldwide. The progestin component is primarily responsible for the contraceptive effect, while the main function of the estrogen component is to balance the impact of the progestin on the endometrium, thereby providing an acceptable bleeding pattern. Most COCs contain ethinyl estradiol (EE) as the estrogen component, in combination with a progestin, which may vary from product to product.

Early EE-containing COCs were associated with medical risks such as a significant increase in venous thromboembolism (VTE) incidence. To reduce these risks, researchers and pharmaceutical companies worked to lower the EE dose or replace it with naturally-occurring estrogens, including estradiol (E2) or E2 valerate (E2V). However, the use of a different estrogen can impact the bleeding profile. For example, COCs containing doses of ≤20 μg EE are generally associated with a less favourable bleeding profile. Currently used COCs such as -E2/NOMAC and E2V with dienogest (E2V/DNG) have a relatively high rate of absence of scheduled bleeding/spotting, ranging from 18-31% over cycles with E2/NOMAC to 19-24% over cycles with E2V/DNG.

COCs comprising drospirenone (DRSP) as the progestin component and estetrol (E4) as the estrogen component have been described in, for example, U.S. Pat. No. 7,732,430. It has been reported that when E4 is used with 3 mg DRSP, the bleeding profile and cycle control is improved in comparison to other COCs using E2V or E2. For example, Apter et al., Contraception, 2016, 94 (4): 366-73, evaluated the bleeding pattern and cycle control of different E4/DRSP or E4/LNG combinations in comparison to a marketed quadriphasic COC containing E2V and dienogest (DNG). The combination of 15 mg E4/DRSP and the combination 20 mg E4/LNG (levonorgestrel) were both associated with a lower incidence of unscheduled bleeding days than the comparator after 6 treatment cycles. In addition, absence of withdrawal bleeding (i.e., absence of scheduled bleeding, also called amenorrhea) was lower with the E4-containing preparations, and particularly with the E4/DRSP combination, than with the comparator. In the reported study, the mean number of days with unscheduled bleeding/spotting by cycle was lower with the 15 mg E4/DRSP as compared to the E2V/DNG combination.

However, the bleeding effects of E4/DRSP on women having different BMI levels have not heretofore been studied in detail, and contraceptive methods with scheduled bleeding or with reduced likelihood of scheduled bleeding based on BMI using such COCs have not heretofore been described.

There thus remains a need for contraceptive methods with scheduled bleeding and for contraceptive methods with reduced likelihood of scheduled bleeding in women having different BMI levels.

### SUMMARY

The present disclosure provides contraceptive methods with increased likelihood of scheduled bleeding in a woman having a BMI<30.0 kg/m² and contraceptive methods with reduced likelihood of scheduled bleeding in a woman having a BMI≥30.0 kg/m².

In one aspect, the present disclosure provides methods of providing contraception with an increased likelihood of scheduled bleeding in a woman having a BMI<30.0 kg/m², comprising: selecting a woman determined to have a BMI<30.0 kg/m², and then orally administering to the selected woman a therapeutically effective amount of an estetrol component at a daily dose of from 14 mg to 16 mg, based on the estetrol moiety, and drospirenone at a daily dose of from 2.5 mg to 3.5 mg, wherein the method comprises administration according to consecutive 28-day cycles of daily administration of the estetrol component and drospirenone on consecutive days 1-24 followed by a hormone-free period without administration of the estetrol component and drospirenone on consecutive days 25-28, wherein the treated woman has an increased likelihood of scheduled bleeding on any of day 25 to day 3 of consecutive 28-day cycles.

In another aspect, the present disclosure provides compositions for oral administration comprising an estetrol component and drospirenone for use in providing contraception with an increased likelihood of scheduled bleeding in a woman having a BMI<30.0 kg/m², wherein the composition comprises from 14 mg to 16 mg of the estetrol component, based on the estetrol moiety, and from 2.5 mg to 3.5 mg drospirenone, wherein the use comprises selecting a woman determined to have a BMI<30.0 kg/m², and then orally administering the composition to the selected woman according to consecutive 28-day cycles of daily administration of the composition on consecutive days 1-24 followed by a hormone-free period without administration of the composition on consecutive days 25-28, wherein the treated woman has an increased likelihood of scheduled bleeding on any of day 25 to day 3 of consecutive 28-day cycles.

In some embodiments of any of the foregoing aspects, the estetrol component is estetrol monohydrate. In some embodiments of any of the foregoing aspects, the estetrol component is estetrol monohydrate and is administered at a daily dose of 15 mg and the drospirenone is administered at a daily dose of 3 mg. In some embodiments of any of the foregoing composition aspects, the estetrol component is estetrol monohydrate and is present in the composition in an amount of 15 mg and the drospirenone is present in an amount of 3 mg.

In some embodiments of any of the foregoing aspects, the methods or uses may further comprise, prior to the selecting, calculating the BMI of the woman from a measured height and weight of the woman.

In some embodiments of any of the foregoing aspects, the selecting comprises selecting a woman determined to have a BMI<30.0 kg/m² and determined to be 16-50 years old. In some embodiments of any of the foregoing aspects, the selecting comprises selecting a woman determined to have a BMI<30.0 kg/m² and determined to be 16-25 years old.

In some embodiments of any of the foregoing aspects, the treated woman has an increased likelihood of experiencing scheduled bleeding on day 25 to day 3 of a 28-day cycle than a woman having a BMI≥30.0 kg/m².

In another aspect, the present disclosure provides methods of providing contraception with reduced likelihood of scheduled bleeding in a woman in a woman having a BMI≥30.0 kg/m², comprising: selecting a woman determined to have a BMI≥30.0 kg/m², and then orally administering to the selected woman a therapeutically effective amount of an estetrol component at a daily dose of from 14 mg to 16 mg, based on the estetrol moiety, and drospirenone at a daily dose of from 2.5 mg to 3.5 mg, wherein the method comprises administration according to consecutive 28-day cycles of daily administration of the estetrol component and drospirenone on consecutive days 1-24 followed by a hormone-free period without administration of the estetrol component and drospirenone on consecutive days 25-28, wherein the woman has a reduced likelihood of scheduled bleeding on day 25 to day 3 of consecutive 28-day cycles.

In another aspect, the present disclosure provides compositions for oral administration comprising an estetrol component and drospirenone for use in providing contraception with reduced likelihood of scheduled bleeding in a woman having a BMI≥30.0 kg/m², wherein the composition comprises from 14 mg to 16 mg of the estetrol component, based on the estetrol moiety, and from 2.5 mg to 3.5 mg drospirenone, wherein the use comprises selecting a woman determined to have a BMI≥30.0 kg/m², and then orally administering the composition to the selected woman according to consecutive 28-day cycles of daily administration of the composition on consecutive days 1-24 followed by a hormone-free period without administration of the composition on consecutive days 25-28, wherein the treated woman has a reduced likelihood of scheduled bleeding on day 25 to day 3 of consecutive 28 day cycles.

In some embodiments of the foregoing aspects, the estetrol component is estetrol monohydrate. In some embodiments of any of the foregoing aspects, the estetrol component is estetrol monohydrate and is administered at a daily dose of 15 mg and the drospirenone is administered at a daily dose of 3 mg. In some embodiments of any of the foregoing composition aspects, the estetrol component is estetrol monohydrate and is present in the composition in an amount of 15 mg and the drospirenone is present in an amount of 3 mg.

In some embodiments of the foregoing aspects, the methods or uses may further comprise, prior to the selecting, calculating the BMI of the woman from a measured height and weight of the woman.

In some embodiments of the foregoing aspects, the selecting comprises selecting a woman determined to have a BMI≥30.0 kg/m² and ≤35.0 kg/m². In some embodiments of the foregoing aspects, the selecting comprises selecting a woman determined to have a BMI≥30.0 kg/m² and determined to be 16-50 years old. In some embodiments of the foregoing aspects, the selecting comprises selecting a woman determined to have a BMI≥30.0 kg/m² and determined to be 16-25 years old.

In some embodiments of the foregoing aspects, the treated woman has a reduced likelihood of experiencing scheduled bleeding on day 25 to day 3 of a 28-day cycle than a woman having a BMI<30.0 kg/m².

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the disposition of participants used for the pooled bleeding analysis of methods of providing contraception using E4/DRSP reported herein.

FIG. 2 shows the percentage of participants reporting scheduled bleeding/spotting, unscheduled bleeding/spotting, and absence of any bleeding/spotting per cycle during use of E4/DRSP oral contraception.

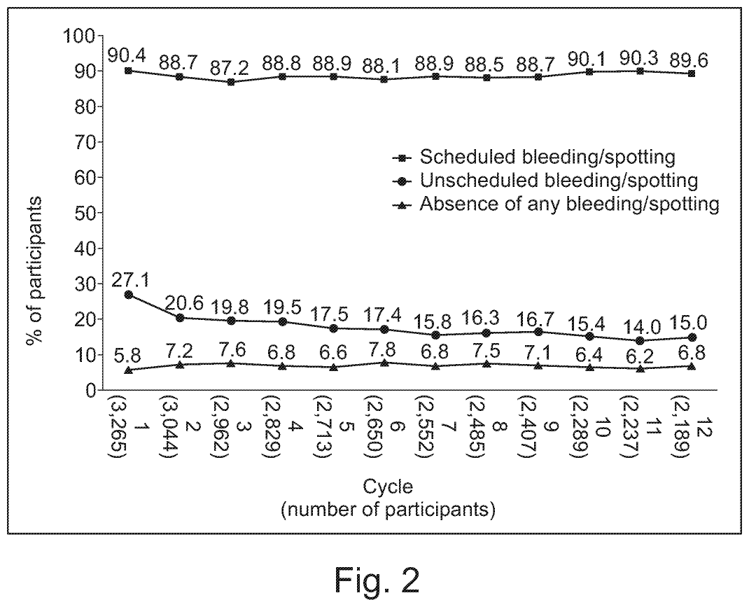

FIG. 3 shows the percentage of participants with unscheduled bleeding/spotting episodes during use of E4/DRSP oral contraception.

FIG. 4 shows the percentage of participants with absence of scheduled bleeding by obesity status (<30.0 kg/m² vs ≥30.0 kg/m²).

### DETAILED DESCRIPTION

The present inventors surprisingly discovered that contraceptive methods using E4/DRSP are associated with different bleeding effects depending on the BMI of the treated woman.

Thus, described herein in accordance with some embodiments are methods of providing contraception with an increased likelihood of scheduled bleeding in a woman having a BMI<30.0 kg/m², comprising selecting a woman determined to have a BMI<30.0 kg/m², and then orally administering to the selected woman a therapeutically effective amount of an estetrol component at a daily dose of from 14 mg to 16 mg, based on the estetrol moiety, and drospirenone at a daily dose of from 2.5 mg to 3.5 mg. Also described are compositions for oral administration comprising an estetrol component and drospirenone for use in providing contraception with an increased likelihood of scheduled bleeding in a woman having a BMI<30.0 kg/m², wherein the composition comprises from 14 mg to 16 mg of the estetrol component, based on the estetrol moiety, and from 2.5 mg to 3.5 mg drospirenone, wherein the use comprises selecting a woman determined to have a BMI<30.0 kg/m², and then orally administering the composition to the selected woman according to consecutive 28-day cycles of daily administration of the composition on consecutive days 1-24 followed by a hormone-free period without administration of the composition on consecutive days 25-28, wherein the treated woman has an increased likelihood of scheduled bleeding on any of day 25 to day 3 of consecutive 28-day cycles.

Also described herein in accordance with other embodiments, are methods of providing contraception with reduced likelihood of scheduled bleeding in a woman in a woman having a BMI≥30.0 kg/m², comprising selecting a woman determined to have a BMI≥30.0 kg/m², and then orally administering to the selected woman a therapeutically effective amount of an estetrol component at a daily dose of from 14 mg to 16 mg, based on the estetrol moiety, and drospirenone at a daily dose of from 2.5 mg to 3.5 mg. Also described are compositions for oral administration comprising an estetrol component and drospirenone for use in providing contraception with reduced likelihood of scheduled bleeding in a woman having a BMI≥30.0 kg/m², wherein the composition comprises from 14 mg to 16 mg of the estetrol component, based on the estetrol moiety, and from 2.5 mg to 3.5 mg drospirenone, wherein the use comprises selecting a woman determined to have a BMI≥30.0 kg/m², and then orally administering the composition to the selected woman according to consecutive 28-day cycles of daily administration of the composition on consecutive days 1-24 followed by a hormone-free period without administration of the composition on consecutive days 25-28, wherein the treated woman has a reduced likelihood of scheduled bleeding on day 25 to day 3 of consecutive 28 day cycles.

### Definitions

As used herein, “about” when used in conjunction with a measurable value such as a parameter, an amount, a temporal duration, and the like, is meant to encompass variations of up to +/−10% from the specified value. Thus, “about 10” should be understood to include any value from 9 to 11, as well as a value of 10.

As used herein, “effective amount” refers to an amount effective to achieve an intended physiological effect, such as contraception. The physiological effect may not be achieved by a single dose and may be achieved by repeated doses, each considered to be “effective.” In the context of contraception “an effective amount” refers to an amount which is effective to suppress ovulation when administered in accordance with an effective regimen. It is emphasized that an “effective amount” will not always achieve the intended physiological effect in every individual subject, even though such dose is deemed to be a therapeutically effective amount by those of skill in the art.

As used herein, “BMI” stands for Body Mass Index, and is defined as body mass of a human subject divided by the square of body height of the human subject, and is expressed in units of kg/m², resulting from mass in kilograms and height in meters.

As used herein, “unscheduled bleeding” refers to vaginal bleeding and/or spotting occurring on Day 4 through Day 24 of a 28-day cycle as described herein. Conversely, as used herein, “scheduled bleeding” refers to bleeding and/or spotting occurring on any of Day 25 of one 28-day cycle up to Day 3 of the next consecutive 28-day cycle and includes any bleeding and/or spotting starting prior to Day 25 and continuing into the scheduled bleeding period, or bleeding and/or spotting that started in the scheduled bleeding period but continued after Day 3.

As used here “spotting” refers to minimal vaginal blood loss that does not require any sanitary protection, including pantyliners.

As used herein a woman is a “switcher” when she discontinues the use of one type of hormonal contraceptive to initiate the use of another type of hormonal contraceptive. Optionally, this switch comprises a break in use of hormonal contraceptives of 4 weeks or more. A woman is typically considered as a “switcher” if previous hormonal contraceptive use took place within 3 months prior to E4/DRSP use.

As used herein a woman is a “true new user” when she is in her first year of ever using a hormonal contraceptive.

As used herein a woman is a “starter” when she has discontinued use of hormonal contraceptives for three months or longer.

As noted above, the methods described herein comprise orally administering an estetrol component and drospirenone and the compositions for use described herein are compositions for oral administration comprising an estetrol component and drospirenone for use in providing contraception.

### Estetrol

The term “estetrol component” as used herein encompasses estetrol, hydrates thereof, esters thereof, and combinations of any thereof.

The chemical name for estetrol is estra-1,3,5 (10)-triene-3, 15α, 16α, 17β-tetrol. Estetrol has a molecular formula of C₁₈H₂₄O₄ and a molecular weight of 304.4 g/mol. Estetrol has the following chemical structure:

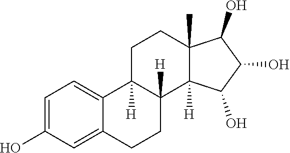

Examples of suitable estetrol esters include esters wherein the hydrogen atom of at least one of the hydroxyl groups of estetrol has been substituted by an acyl radical of a hydrocarbon carboxylic, sulfonic acid or sulfamic acid of 1-25 carbon atoms.

In some embodiments, the estetrol component is estetrol monohydrate. The chemical name for estetrol monohydrate is estra-1,3,5 (10)-triene-3,15α, 16α, 17α-tetrol monohydrate. It has a molecular formula of C₁₈H₂₄O₄⋅H₂O and a molecular weight of 322.4 g/mol, equivalent to 304.4 g/mol (anhydrous). Estetrol monohydrate has the following chemical structure:

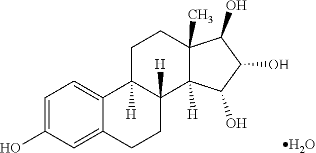

Estetrol monohydrate is a white to off-white crystalline solid that is poorly soluble in water and aqueous solutions. It is soluble in methanol, ethanol, sparingly soluble in acetone, and slightly soluble in ethyl acetate and acetonitrile.

### Drospirenone

Drospirenone has the chemical name (6R,7R,8R,9S,10R,13S,14S,15S,16S,17S) 1,3′, 4′, 6,6a, 7,8,9,10,11,12,13,14,15,15a, 16-hexadecahydro10,13-dimethylspiro-[17Hdicyclopropa-[6,7:15,16] cyclopenta[a]phenanthrene-17,2′ (5H)-furan]-3,5′ (2H)-dione). It has a molecular weight of 366.5 g/mol, a molecular formula of C₂₄H₃₀O₃, and the following chemical structure:

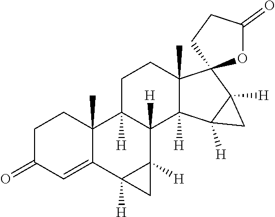

Drospirenone is a white to almost white or slightly yellow crystalline powder. It is a neutral molecule with slight solubility in water.

### Contraceptive Methods and Bleeding Effects

As noted above, the present inventors surprisingly discovered that contraceptive methods using E4/DRSP are associated with different bleeding effects depending on the BMI of the treated woman. In particular, methods of providing contraception in a woman having a BMI<30.0 kg/m² as described herein and compositions for oral administration comprising an estetrol component and drospirenone for use in providing contraception in a woman having a BMI<30.0 kg/m² as described herein are associated with an increased likelihood of scheduled bleeding, while methods of providing contraception in a woman having a BMI≥30.0 kg/m² as described herein and compositions for oral administration comprising an estetrol component and drospirenone for use in providing contraception in a woman having a BMI≥30.0 kg/m² as described herein are associated with a reduced likelihood of scheduled bleeding.

Analysis of pooled bleeding data from two parallel, open-label, 13-cycle phase 3 clinical trials discussed in more detail below surprisingly showed that absence of scheduled bleeding/spotting occurred more frequently in women with a BMI≥30.0 kg/m² (13.4-21.6% per cycle) than women with a BMI<30.0 kg/m² (7.6-9.0% per cycle) (OR 1.68, 95% CI 1.37-2.05). On the other hand, no difference was found in unscheduled bleeding/spotting when stratified by BMI (OR 1.16 95% CI 0.98-1.37). In other words, women with a BMI≥30.0 kg/m² that are treated with a combination of E4/DRSP have a reduced likelihood of scheduled bleeding compared to women with a BMI<30.0 kg/m², and women with a BMI<30.0 kg/m² have an increased likelihood of scheduled bleeding, and generally experience scheduled bleeding. Prior to the present invention, the differential effect on bleeding patterns of E4/DRSP based on BMI was not known.

For either patient population, the methods and uses of E4/DSRP described herein comprise orally administering to the woman a therapeutically effective amount of an estetrol component at a daily dose of from 14 mg to 16 mg, based on the estetrol moiety, and drospirenone at a daily dose of from 2.5 mg to 3.5 mg. The compositions described herein are compositions for oral administration comprising an estetrol component and drospirenone, comprising from 14 mg to 16 mg of the estetrol component, based on the estetrol moiety, and from 2.5 mg to 3.5 mg drospirenone, In specific embodiments, the estetrol component is estetrol monohydrate. In further specific embodiments, the estetrol component is estetrol monohydrate and is administered at a daily dose of 15 mg and the drospirenone is or administered at a daily dose of 3 mg. Thus, a composition for use as described herein may comprise estetrol monohydrate as the estetrol component. Optionally, the estetrol monohydrate is present in the composition in an amount of 15 mg and the drospirenone is present in the composition in an amount of 3 mg.

As discussed in more detail below, the estetrol component and drospirenone may be administered in separate dosage units, or in a single dosage unit comprising both the estetrol component and drospirenone. In specific embodiments, a composition as described herein comprises both an estetrol component and drospirenone.

The uses and methods described herein typically will comprise administration according to consecutive 28-day cycles of daily administration of the estetrol component and drospirenone (e.g., daily oral administration of a composition as described herein comprising an estetrol component and drospirenone) on consecutive Days 1-24 followed by a hormone-free period without administration of the estetrol component and drospirenone (e.g., without administration of the composition) on consecutive Days 25-28.

For uses and methods of providing contraception with scheduled bleeding in women having a BMI<30.0 kg/m², the uses and methods comprise selecting a woman determined to have a BMI<30.0 kg/m² and then orally administering to the selected woman a therapeutically effective amount of an estetrol component at a daily dose of from 14 mg to 16 mg, based on the estetrol moiety, and drospirenone at a daily dose of from 2.5 mg to 3.5 mg. For such uses, a composition as described herein may be a composition for oral administration comprising an estetrol component and drospirenone, comprising from 14 mg to 16 mg of the estetrol component, based on the estetrol moiety, and from 2.5 mg to 3.5 mg drospirenone. In specific embodiments, the estetrol component is estetrol monohydrate. In further specific embodiments, the estetrol component is estetrol monohydrate and is administered at a daily dose of 15 mg and the drospirenone is administered at a daily dose of 3 mg. Thus, a composition for use as described herein may comprise estetrol monohydrate as the estetrol component. Optionally, the estetrol monohydrate is present in the composition in an amount of 15 mg and the drospirenone is present in the composition in an amount of 3 mg.

In accordance with such uses and methods, the treated woman having a BMI<30.0 kg/m² experiences scheduled bleeding on any of Day 25 to Day 3 of consecutive 28-day cycles (i.e., on any of Day 25-Day 28 of one 28-day cycle and/or any of Day 1 to Day 3 of the next consecutive 28-day cycle).

For women having a BMI<30.0 kg/m², scheduled bleeding may occur at a rate of at least 90% per cycle, at least 91% per cycle, at least 92% per cycle, at least 93% per cycle, at least 94% per cycle, at least 95% per cycle, at least 96% per cycle, at least 97% per cycle, at least 98% per cycle, at least 99% per cycle, or 100% per cycle. Additionally or alternatively, for women having a BMI<30.0 kg/m², scheduled bleeding may be about 1.50 times more likely, about 1.55 times more likely, about 1.60 times more likely, about 1.65 times more likely, about 1.70 times more likely, or about 1.75 times more likely, as compared to women with a BMI≥30.0 kg/m².

In some embodiments, the uses and methods further comprise, prior to the selecting, calculating the BMI of the woman from a measured height and weight of the woman. Calculating the BMI can include measuring the woman's height and weight.

In some embodiments, the selecting comprises selecting a woman determined to have a BMI<30.0 kg/m² and determined to be 16-50 years old.

In some embodiments, the selecting comprises selecting a woman determined to have a BMI<30.0 kg/m² and determined to be 16-25 years old.

For uses and methods of providing contraception with reduced likelihood of scheduled bleeding in women having a BMI≥30.0 kg/m², the uses and methods comprise selecting a woman determined to have a BMI≥30.0 kg/m², and then orally administering to the selected woman a therapeutically effective amount of an estetrol component at a daily dose of from 14 mg to 16 mg, based on the estetrol moiety, and drospirenone at a daily dose of from 2.5 mg to 3.5 mg. For such uses, a composition as described herein may be a composition for oral administration comprising an estetrol component and drospirenone, comprising from 14 mg to 16 mg of the estetrol component, based on the estetrol moiety, and from 2.5 mg to 3.5 mg drospirenone, In specific embodiments, the estetrol component is estetrol monohydrate. In further specific embodiments, the estetrol component is estetrol monohydrate and is administered at a daily dose of 15 mg and the drospirenone is administered at a daily dose of 3 mg. Thus, a composition for use as described herein may comprise estetrol monohydrate as the estetrol component. Optionally, the estetrol monohydrate is present in the composition in an amount of 15 mg and the drospirenone is present in the composition in an amount of 3 mg.

In accordance with such uses and methods, the treated woman having a BMI≥30.0 kg/m² has a reduced likelihood of scheduled bleeding on Day 25 to Day 3 of consecutive 28-day cycles (i.e., has a reduced likelihood of scheduled bleeding on Day 25-Day 28 of one 28-day cycle and Day 1 to Day 3 of the next consecutive 28 day cycle). For example, the woman may have a reduced likelihood of experiencing scheduled bleeding on day 25 to day 3 of a 28-day cycle than a woman having a BMI<30.0 kg/m².

For women having a BMI≥30.0 kg/m², scheduled bleeding may occur at a rate of less than 90% per cycle, less than 89% per cycle, less than 88% per cycle, less than 87% per cycle, less than 86% per cycle, less than 85% per cycle, less than 84% per cycle, less than 83% per cycle, less than 82% per cycle, less than 81% per cycle, or less than 80% per cycle. Additionally or alternatively, for women having a BMI≥30.0 kg/m², scheduled bleed may occur about 4%, about 5%, about 6%, about 7%, about 8%, about 9%, about 10%, about 11%, about 12%, about 13%, about 14%, or about 15% less often as compared to women with a BMI<30.0 kg/m². Additionally or alternatively, for the purposes of treating women having a BMI≥30.0 kg/m², the likelihood of scheduled bleeding is about 1.50 times less, about 1.55 times less, about 1.60 times less, about 1.65 times less, about 1.70 times less, or about 1.75 times less compared to women with a BMI<30.0 kg/m².

In some embodiments, the uses and methods further comprise, prior to the selecting, calculating the BMI of the woman from a measured height and weight of the woman.

In some embodiments, the selecting comprises selecting a woman determined to have a BMI≥30.0 kg/m² and determined to be 16-50 years old.

In some embodiments, the selecting comprises selecting a woman determined to have a BMI≥30.0 kg/m² and determined to be 16-25 years old.

While the uses and methods described herein contemplate daily administration of the estetrol component and drospirenone (e.g., daily oral administration of a composition as described herein comprising an estetrol component and drospirenone) on consecutive Days 1-24 followed by a hormone-free period without administration of the estetrol component and drospirenone (e.g., without administration of the composition) on consecutive Days 25-28, it should be understood that minor interruptions in the administration schedule may occur without significantly impacting the overall contraceptive effectiveness, and such aberrations are encompassed by the present disclosure. In general, if one administration is missed, the missed dosage form(s) should be taken as soon as possible and the next administration should still occur at the regularly scheduled time, even if this results in two administrations in a single day.

In order to achieve maximum effectiveness for contraception, it may be advisable to administer the dosage form(s) comprising the estetrol component and drospirenone (e.g., a composition as described herein comprising an estetrol component and drospirenone) at around the same time each day. Further, the first administration (i.e., Day 1 of the first treatment cycle) is typically administered on the first day of menstruation, or, in the case of a switcher, on the first day when a new cycle of treatment for the woman's prior oral contraceptive would have started.

In some embodiments, the hormone-free period (without administration of the estetrol component and drospirenone or composition as described herein) on, for example, consecutive Days 25-28, may comprise taking a dosage form (e.g., pill, tablet, etc.) that does not contain an estetrol component or drospirenone (e.g., an inert pill, tablet, etc.).

The woman being administered the contraceptive (i.e., the treated woman) may be a true new user (i.e., she has not previously used an oral contraceptive), a switcher, or a starter.

### Oral Pharmaceutical Compositions

Oral pharmaceutical compositions useful for carrying out the uses and methods described herein are not particularly limited; suitable non-limiting examples are described herein.

As noted above, the estetrol component and drospirenone may be formulated in separate compositions or in a single composition comprising both the estetrol component and drospirenone. Typically, the estetrol component and drospirenone are formulated in oral dosage units that provide a daily dose of one or both of the estetrol component and drospirenone, such as from 14 mg to 16 mg of the estetrol component, based on the estetrol moiety, and 2.5 to 3.5 mg of drospirenone. In specific embodiments, the estetrol component and drospirenone are formulated together in oral dosage units that provide a daily dose of both the estetrol component and drospirenone, such as from 14 mg to 16 mg of the estetrol component, based on the estetrol moiety, and 2.5 to 3.5 mg of drospirenone. In further specific embodiments, the estetrol component is estetrol monohydrate and the estetrol monohydrate and drospirenone are formulated together in oral dosage units that contain 15 mg of estetrol monohydrate and 3 mg of drospirenone. Thus, a composition as described herein may be in the form of an oral dosage unit that provides a daily dose of both the estetrol component and drospirenone, such as from 14 mg to 16 mg of the estetrol component, based on the estetrol moiety, and 2.5 to 3.5 mg of drospirenone, such as 15 mg of estetrol monohydrate and 3 mg of drospirenone.

The oral dosage units may be in the form of tablets, capsules, cachets, pellets, pills, powders, or granules capsules comprising a dry composition, or capsules that contain a liquid composition, such as an oil, in which the estetrol component is dissolved or dispersed. Tablets, pellets, and pills can, optionally, be film-coated to achieve a desired release profile.

Dry composition(s) may further comprise one or more pharmaceutically acceptable excipients such as, but not limited to, one or more binders (e.g. hydroxypropylmethyl cellulose, polyvinyl pyrrolidone, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc), etc. For example, pharmaceutical excipients or inactive ingredients that may be used to prepare a COC dosage form as described herein can include, but are not limited to, corn starch, lactose monohydrate, magnesium stearate, povidone, and sodium starch glycolate. Dry composition(s) may be prepared by wet granulation, using an aqueous solution or an organic solution, or by direct compression.

Optionally, a composition as described herein is in the form of a tablet comprising, in addition to the estetrol component and drospirenone, a filler/diluent, a superdisintegrant, a binder and disintegrant, a further binder, and a lubricant. In preferred embodiments, the composition is in the form of a tablet comprising the estetrol component, drospirenone, lactose, sodium starch glycolate, maize/corn starch, povidone, and magnesium stearate. Preferably, the composition is in the form of a tablet comprising estetrol monohydrate, drospirenone, lactose monohydrate, sodium starch glycolate type A, maize starch, povidone K30, and magnesium stearate. Optionally, the tablet is coated with a coating agent. In further optional embodiments, the coating agent comprises hypromellose, hydroxypropylcellulose, titanium dioxide, red iron oxide, hydrogenated cottonseed oil, and talc. By means of illustration and not limitation, a suitable coating agent is AquaPolish Pink 044.08 MS.

Dry or liquid composition(s) may further comprise one more pharmaceutically acceptable excipients such as, but not limited to, one or more of antioxidants, stabilizers, preservatives, coloring agents, flavouring agents, etc.

Typically, any excipients present in an oral dosage unit will adhere to pharmaceutical grade industry quality standards such as Ph. Eur. and USP-NF.

As noted above, the estetrol component may be one or more substances selected from estetrol, hydrates of estetrol, and esters of estetrol. In specific embodiments, the estetrol component is estetrol monohydrate.

In some embodiments, the amount of the estetrol component may be 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, or 16 mg, based on the estetrol moiety. In some embodiments, the amount of the estetrol component is 14.2 mg based on the estetrol moiety. When the estetrol component is estetrol monohydrate, 15 mg estetrol monohydrate may provide 14.2 mg based on the estetrol moiety.

In some embodiments, the amount of drospirenone may be 2.5, 2.6, 2.7, 2.8, 2.9, 3.0, 3.1, 3.2, 3.3, 3.4, or 3.5 mg. In some embodiments, the amount of drospirenone is 3.0 mg.

For convenience, the dosage forms necessary to complete a 28-day treatment cycle may be provided in a single package, such as a single blister pack containing (i) 24 active tablets comprising an estetrol component and drospirenone (e.g., a composition as described herein comprising an estetrol component and drospirenone), and (ii) 4 inert tablets that do not contain an estetrol component and that do not contain drospirenone.

The uses and methods and compositions of the present disclosure have been described above with reference to a number of exemplary embodiments. Modifications and alternative implementations of some parts or elements are possible, and are included in the scope of the disclosure. The following examples are provided to illustrate certain embodiments of the disclosed uses and methods and compositions. It should be understood, however, that the invention is not limited to the specific conditions or details described in these examples.

### Example 1—Pooled Bleeding Analysis

Overview: Pooled bleeding data from two parallel, open-label, 13-cycle phase 3 clinical trials that enrolled 3,409 participants 16-50 years old with body mass index (BMI)≤35.0 kg/m² was analysed to evaluate the bleeding patterns of a combined oral contraceptive (COC) containing estetrol (E4) and drospirenone in a 24/4-day regimen. Scheduled bleeding/spotting occurred in 87.2-90.4% of participants per cycle, with a median duration of 4-5 days. Unscheduled bleeding/spotting decreased from 27.1% in Cycle 1 to 20.6% in Cycle 2 to ≤17.5% from Cycle 5 onwards. Most (66.5%) unscheduled bleeding/spotting episodes were spotting-only. Between 5.8-7.8% of users per cycle experienced absence of any scheduled or unscheduled bleeding/spotting (amenorrhea). Participants with a BMI≥30.0 kg/m² reported more absence of scheduled bleeding/spotting (OR 1.68 [1.37-2.05]).

The purpose of this example was to evaluate the bleeding patterns of a combined oral contraceptive containing estetrol (E4) monohydrate 15 mg/drospirenone (DRSP) 3 mg in a 24/4-day regimen. Bleeding data was pooled from two parallel, open-label, 13-cycle phase 3 trials that enrolled participants 16-50 years old with body mass index (BMI)≤35.0 kg/m². Participants reported vaginal bleeding/spotting in daily diaries. For this bleeding analysis, participants with at least one evaluable cycle were included. The mean frequencies of scheduled and unscheduled bleeding/spotting episodes and the median duration of bleeding/spotting episodes were calculated, and the associations between treatment compliance, BMI, and recent hormonal contraceptive use were assessed with respect to impact on bleeding/spotting outcomes.

As further detailed below, bleeding patterns of E4/DRSP users were unexpectedly different depending on the BMI of the woman being treated. The present invention therefore provides for the first time an ability to advise a woman of the expected bleeding pattern she is more likely to experience (based on her BMI), which may improve the patient experience, improve compliance, and promote continuation of treatment.

### Materials and Methods

This pooled analysis included bleeding data from two multi-center, open-label, non-comparator phase 3 clinical trials that evaluated contraceptive efficacy, bleeding patterns, and safety associated with the use of E4 15 mg/DRSP 3 mg oral contraceptive pills.

The studies enrolled healthy heterosexually active, pre-menopausal participants (aged 16-50 years in the U.S./Canada trial; aged 18-50 years in the Europe/Russia trial) with a BMI of 18.0-35.0 kg/m² and regular menstrual cycles, according to previously described inclusion and exclusion criteria (Creinin et al., Contraception, 2021, 104(3):222-228 and Danielsson et al., BJOG, 2022, 129(1):63-71). Participants could have switched from a previous hormonal contraceptive method except for injectable contraceptives. Eligible participants received E4 15 mg (as monohydrate, equivalent to 14.2 mg anhydrous)/DRSP 3 mg once daily in a 24/4-day regimen for up to thirteen 28-day cycles. Switchers from another COC started study treatment when the next pill pack of the previous formulation would have been due, and new users started treatment on the first day of menstruation.

Participants used a daily paper diary to record study medication intake and vaginal bleeding or spotting. Study staff reviewed diaries and asked about adverse events (AEs) at each scheduled follow-up visit during cycles 2, 4, 7, 10, and 13 and, when applicable, during an early discontinuation visit.

Bleeding outcomes were evaluated in participants who started treatment and provided any bleeding outcome data. Bleeding analysis definitions were adopted from the prior art, as summarized below in Table 1.

TABLE 1
Definitions used for analysis of bleeding patterns

| Term | Definition |
|---|---|
| Spotting | Minimal vaginal blood loss that did not require the new use of |
| | sanitary protection, including pantyliners |
| Bleeding | Vaginal blood loss that required the use of sanitary protection |
| | with a tampon, pad or pantyliner |
| Bleeding/spotting episode | One or more consecutive bleeding/spotting days bounded on |
| | either end by two bleeding/spotting free days |
| Scheduled bleeding | Bleeding/spotting from day 25 (first day of hormone free |
| | interval) up to day 3 of the subsequent cycle, including any |
| | bleeding/spotting starting prior to Day 25 and continuing into |
| | the scheduled bleeding period, or bleeding/spotting that started |
| | in the scheduled bleeding period but continued after Day 3 |
| Unscheduled | Any bleeding/spotting not meeting criteria for schedule |
| bleeding/spotting | bleeding |

Participants' diary entries were used to assess pill intake and analyze bleeding patterns by cycle, including frequencies of unscheduled bleeding/spotting episodes and number of unscheduled bleeding/spotting days, frequency of scheduled bleeding/spotting episodes and number of scheduled bleeding/spotting days, frequency of absence of scheduled bleeding and frequency of absence of any bleeding.

For all bleeding assessments, any cycles with a duration >28 days, which occurred when participants skipped one or more days of pills but continued intake once daily through the end of the pack, were excluded from the analysis. The last treatment cycle (Cycle 13) was also excluded because the diaries did not include bleeding data beyond the end of Cycle 13 when, with a 24/4 regimen, the continuation of the scheduled bleeding/spotting episode would have been expected.

Treatment compliance was also assessed based on diary entries. When participants did not complete pill intake information on the diary, that day was designated as a missed pill.

A multivariable longitudinal model was used to assess the association of compliance (0 to ≥5 active pills missed/cycle), BMI (<30.0 kg/m², ≥30.0 kg/m²) and hormonal contraceptive use during the 3 months prior to enrollment (starters vs. switchers), with the absence of scheduled bleeding and unscheduled bleeding/spotting by cycle. A generalized linear mixed model was fitted with participants as repeated random effect (first-order autoregressive correlation structure) on the occurrence of absent scheduled bleeding and unscheduled bleeding across valid cycles 2 through 12. Statistical analyses were performed using SAS® software (version 9.4) for Windows®.

### Results: Participants and Compliance

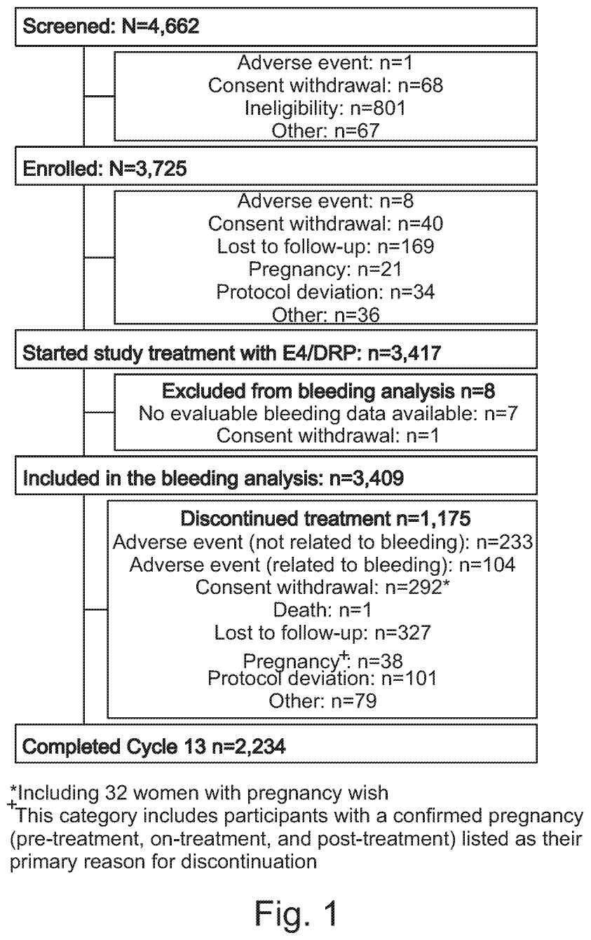

Among 3,725 enrolled participants, 3,417 had confirmed initiation of E4/DRSP treatment; 7 participants did not provide evaluable bleeding data and one participant withdrew consent, leaving 3,409 (99.8%) with a total of 33,815 evaluable cycles in the analysis population (FIG. 1). The demographic and baseline characteristics of the participants are presented in Table 2.

TABLE 2
Demographic and baseline characteristics of participants treated
with E4/DRSP in a pooled analysis of two phase 3 clinical trials

| | Pooled population | |
|---|---|---|
| Characteristic | n = 3,417 | |
| Age (years) | 27.2 ± 6.7 | |
| BMI (kg/m²) | 24.6 ± 4.4 | |
| <30.0 | 2,896 | (84.8) |
| ≥30.0 | 521 | (15.2) |
| Current smoker^{†} | 468 | (13.7) |
| Nulligravid | 2,027 | (59.3) |
| Nulliparous | 2,265 | (66.3) |
| Previous hormonal contraceptive use^{‡} | | |
| Switchers^{‡} | 1,732 | (50.7) |
| Starters^{§} | 1,685 | (49.3) |
| True new users | 674 | (19.7) |
| Race | | |
| Asian | 97 | (2.8) |
| Black | 377 | (11.0) |
| White | 2,832 | (82.9) |
| Other^{¥} | 111 | (3.3) |

Data are presented as n (%) or mean ± standard deviation
^{†}Current smokers aged >35 years were excluded
^{‡}Switchers: previous hormonal contraceptive use within 3 months before initiating study drug
^{§}Starters: previous hormonal contraceptive use >3 months before initiating study drug (starters) and none (true new users)
^{¥}Includes America Indian or Alaska Native, Native Hawaiian, or other Pacific Islanders, and Other

Overall, 2,234 (65.4%) participants completed 13 treatment cycles. One hundred and four (3%) participants discontinued due to bleeding-related adverse events. The most frequently reported bleeding-related AEs (in ≥2% of participants) included irregular bleeding (MedDRA term: metrorrhagia, 4.7%), vaginal bleeding (MedDRA term: vaginal hemorrhage, 3.0%) and dysmenorrhea (2.5%).

Most (>82%) participants did not report missing any pills. The proportion of participants who missed one pill ranged from 9.3% (Cycle 1) to 5.4% (Cycles 12 and 13), who missed two pills ranged from 3.8% (Cycle 3) to 1.6% (Cycle 11) and who missed more than two pills ranged from 4.6% (Cycle 2) to 1.5% (Cycle 13), respectively.

### Results: Scheduled Bleeding/Spotting

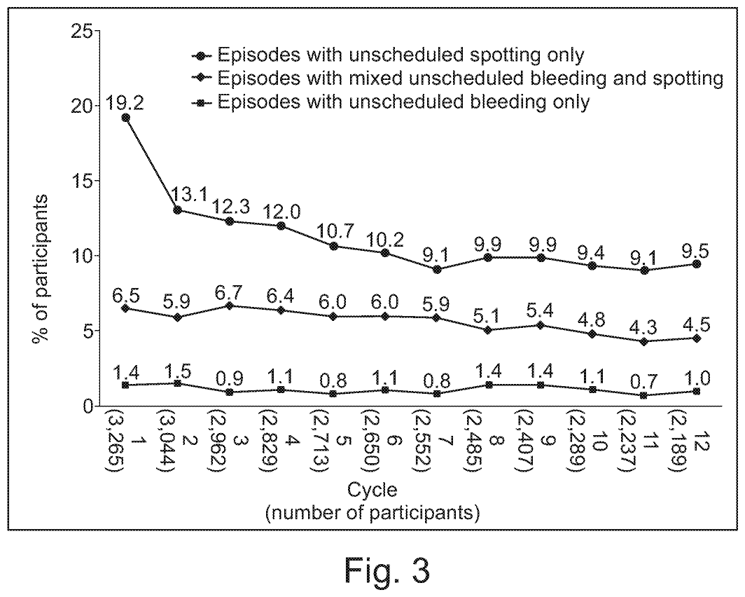

Overall, bleeding and spotting days during the treatment period show a clear cyclic pattern with bleeding at the end of each cycle. Most (87.2% to 90.4%, mean 89.0=0.9%) participants reported scheduled bleeding/spotting across cycles 1-12 (FIG. 3). The number of scheduled bleeding/spotting days remained stable throughout the cycles with a median duration of 4 to 5 days. Scheduled bleeding/spotting consisted of an approximately equal number (2 to 3) of spotting days and bleeding days (Table 3).

TABLE 3
Number of unscheduled and scheduled bleeding/spotting days by cycle

| | | | | |
|---|---|---|---|---|
| | | Number of Bleeding | Number of | Number of |
| | Number of | and Spotting Days | Bleeding Days | Spotting Days |
| Cycle | participants | Median (Q1; Q3) | Median (Q1; Q3) | Median (Q1; Q3) |

Unscheduled bleeding and/or spotting

| | | | | |
|---|---|---|---|---|
| 1 | 886 | 3 (2; 6) | 0 (0; 1) | 3 (1; 4) |
| 2 | 627 | 3 (2; 5) | 0 (0; 2) | 2 (1; 4) |
| 3 | 587 | 3 (2; 5) | 0 (0; 3) | 2 (1; 3) |
| 4 | 551 | 3 (2; 5) | 0 (0; 2) | 2 (1; 3) |
| 5 | 476 | 3 (2; 5) | 0 (0; 2) | 2 (1; 4) |
| 6 | 460 | 4 (2; 5) | 0 (0; 2) | 2 (1; 3) |
| 7 | 399 | 4 (2; 5) | 0 (0; 2) | 2 (2; 4) |
| 8 | 406 | 3 (2; 5) | 0 (0; 2) | 2 (1; 3) |
| 9 | 403 | 3 (2; 5) | 0 (0; 2) | 2 (1; 3) |
| 10 | 352 | 3 (2; 5) | 0 (0; 2) | 2 (1; 3) |
| 11 | 314 | 3 (2; 5) | 0 (0; 2) | 2 (1; 3) |
| 12 | 329 | 3 (2; 5) | 0 (0; 2) | 2 (1; 3) |

Scheduled bleeding and/or spotting

| | | | | |
|---|---|---|---|---|
| 1 | 2,951 | 5 (4; 7) | 3 (2; 4) | 2 (1; 4) |
| 2 | 2,700 | 5 (4; 6) | 3 (2; 4) | 2 (1; 3) |
| 3 | 2,583 | 5 (4; 6) | 2 (2; 4) | 2 (1; 3) |
| 4 | 2,513 | 5 (4; 6) | 3 (2; 4) | 2 (1; 3) |
| 5 | 2,413 | 5 (4; 6) | 3 (1; 4) | 2 (1; 3) |
| 6 | 2,334 | 5 (4; 6) | 3 (1; 4) | 2 (1; 3) |
| 7 | 2,241 | 5 (4; 6) | 3 (1; 4) | 2 (1; 3) |
| 8 | 2,199 | 5 (4; 6) | 3 (1; 4) | 2 (1; 3) |
| 9 | 2,135 | 5 (4; 6) | 3 (1; 4) | 2 (1; 3) |
| 10 | 2,063 | 5 (4; 6) | 2 (1; 4) | 2 (1; 3) |
| 11 | 2,021 | 4 (4; 6) | 2 (1; 4) | 2 (1; 3) |
| 12 | 1,961 | 4 (4; 6) | 2 (1; 4) | 2 (1; 3) |

Q1: first quartile;
Q3 third quartile

### Results: Unscheduled Bleeding/Spotting and Absence of Bleeding/Spotting

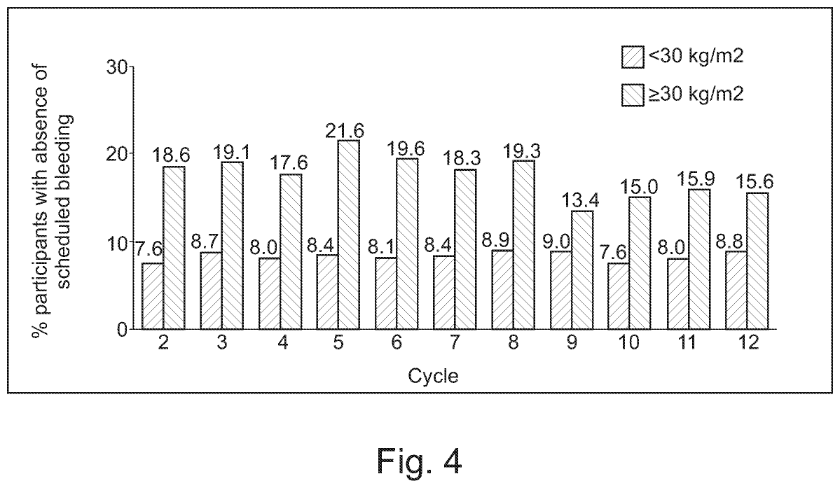

The proportion of participants reporting unscheduled bleeding/spotting episodes decreased from 27.1% in Cycle 1 to 19.5-20.6% during Cycles 2-4 and remained relatively stable thereafter, ranging from 17.5% at Cycle 5 to 14.0% at Cycle 11 (FIG. 3). The mean frequency of unscheduled bleeding/spotting episodes over cycles 1-12 was 17.9%, of which 11.2% was spotting only, 5.6% was bleeding and spotting and 1.1% was bleeding only. With increased duration of use, participants reported less unscheduled spotting-only episodes (from 19.2% in Cycle 1 to 9.5% in Cycle 12), less unscheduled mixed-bleeding/spotting (from 6.5% to 4.5%) and less unscheduled bleeding (from 1.4% to 1.0%) (FIG. 4). Overall, 66.5% of unscheduled bleeding/spotting episodes included only spotting, 26.7% mixed bleeding and spotting, and 6.8% only bleeding. The number of bleeding days was less than 2-fold the number of spotting days (Table 3, above).

The number of unscheduled bleeding or spotting days remained stable throughout the study, with a median duration of 3-4 days among those participants reporting unscheduled bleeding and/or spotting.

Of the 2,234 participants who completed 13 cycles of treatment, 911 (40.8%) reported no unscheduled bleeding/spotting and 754 (33.8%) experienced unscheduled bleeding/spotting in only 1 or 2 cycles.

As shown in FIG. 3, 9.6-12.8% of users per cycle reported no scheduled bleeding. Additionally, 5.8-7.8% (mean 6.9±0.6%) of users across cycles 1-12 reported no bleeding (scheduled or unscheduled).

Participants who reported missing one or more active pills in a cycle, compared to those who missed no pills, more frequently reported unscheduled bleeding/spotting (OR 2.13, 95% CI 1.68-2.70) and absence of scheduled bleeding (OR 2.36, 95% CI 1.82-3.07). The odds increased with the number of missed pills (see Table 4 below).

Prior use of CHC (starters vs switchers) did not affect absence of scheduled bleeding/spotting (OR 1.00, 95% CI 0.85-1.91) or unscheduled bleeding/spotting (OR 0.94 95% CI 0.82-1.07) when analyzed over all cycles, or, as shown in Table 4 below, when analyzed per cycle.

### Results: Bleeding Patterns by BMI

Surprisingly, absence of scheduled bleeding/spotting occurred more frequently in participants with a BMI≥30.0 kg/m² (13.4-21.6% per cycle) than <30.0 kg/m² (7.6-9.0% per cycle) (OR 1.68, 95% CI 1.37-2.05). On the other hand, no difference was found in unscheduled bleeding/spotting when stratified by BMI (OR 1.16 95% CI 0.98-1.37) (Table 4).

Table 4. Absence of scheduled bleeding (Table 4A) and unscheduled bleeding (Table 4B)

TABLE 4A
Absence of scheduled bleeding/spotting

| Parameter | Comparator | Reference | Odds Ratio (95% CI) | P-value |
|---|---|---|---|---|
| Compliance* | ≥1 missed | No missed pills | 2.36 (1.82-3.07) | <.0001 |
| | pills | | | |
| | 1 missed pill | No missed pills | 1.31 (1.00-1.71) | 0.050 |
| | 2 missed pills | No missed pills | 1.82 (1.25-2.64) | 0.002 |
| | 3 missed pills | No missed pills | 1.79 (0.96-3.33) | 0.07 |
| | 4 missed pills | No missed pills | 3.23 (1.60-6.50) | 0.001 |
| | ≥5 missed | No missed pills | 5.35 (2.95-9.72) | <.0001 |
| | pills | | | |
| BMI | ≥30.0 kg/m² | <30.0 kg/m² | 1.68 (1.37-2.05) | <.0001 |
| Previous COC use^{†} | | | | |
| Over all cycles | Starter | Switcher from COC | 1.00 (0.85-1.91) | 0.97 |
| Cycle 2 | Starter | Switcher from COC | 1.01 (0.77-1.31) | 0.97 |
| Cycle 3 | Starter | Switcher from COC | 1.06 (0.83-1.37) | 0.63 |
| Cycle 4 | Starter | Switcher from COC | 0.93 (0.71-1.21) | 0.58 |

TABLE B
Unscheduled bleeding/spotting

| Parameter | Comparator | Reference | Odds Ratio (95% CI) | P-value |
|---|---|---|---|---|
| Compliance* | ≥1 missed | No missed pills | 2.13 (1.68-2.70) | <.0001 |
| | pills | | | |
| | 1 missed pill | No missed pills | 1.40 (1.11-1.77) | 0.005 |
| | 2 missed pills | No missed pills | 1.70 (1.13-2.56) | 0.01 |
| | 3 missed pills | No missed pills | 3.15 (1.87-5.33) | <.0001 |
| | 4 missed pills | No missed pills | 2.44 (1.27-4.71) | 0.01 |
| | ≥5 missed | No missed pills | 2.40 (1.36-4.23) | 0.003 |
| | pills | | | |
| BMI | ≥30.0 kg/m² | <30.0 kg/m² | 1.16 (0.98-1.37) | 0.09 |
| Previous COC use^{†} | | | | |
| Over all cycles | Starter | Switcher from | 0.94 (0.82-1.07) | 0.35 |
| | | COHC | | |
| Cycle 2 | Starter | Switcher from COC | 0.95 (0.79-1.15) | 0.63 |
| Cycle 3 | Starter | Switcher from COC | 1.19 (0.97-1.45) | 0.09 |
| Cycle 4 | Starter | Switcher from COC | 0.98 (0.80-1.19) | 0.83 |

*Based on active pills only
^{†}A separate analysis comparing starters vs switchers from combined oral contraceptives demonstrated similar results (data not shown).

### Discussion

A regular bleeding pattern is an important factor influencing contraceptive selection, adherence, and treatment continuation. Definitions for unscheduled bleeding can vary across studies but absence of scheduled bleeding-spotting is a consistent outcome. Pooled bleeding analyses of phase 3 trials conducted with other COCs demonstrate the frequency of absence of scheduled bleeding/spotting over cycles between 8-12% with EE 20 μg/DRSP 3 mg, 24/4 regimen, 18-32% with E2 1.5 mg/NOMAC 2.5 mg, 24/4 regimen, and 19-24% with E2V 1/2/3 mg/DNG ⅔ mg, 26/2 regimen. The absence of scheduled bleeding/spotting with E4/DRSP (10-13%) appears comparable to EE/DRSP and less frequently compared to E2 formulations. However, these comparisons are indirect. The bleeding profile of E4/DRSP was not impacted by prior use of COCs, not even in the first treatment cycle. With E4/DRSP use, missing one or more pills per cycle increased the frequency of both absence of scheduled bleeding/spotting and of unscheduled bleeding/spotting, and the pattern became less favorable when more pills were missed.

Surprisingly, women with BMI≥30.0 kg/m² (e.g., obese women), experienced more absence of scheduled bleeding (13.4-21.6% per cycle) compared to women with BMI<30.0 kg/m² (e.g., non-obese women) (7.6-9.0% per cycle), as shown in FIG. 4. These results were particularly surprising in view of reports referring to amenorrhea rates with use of a COC containing EE 10 μg and norethindrone acetate 1 mg which demonstrate minimal difference in obese (50.1%), overweight (BMI≥25.0 to <30.0 kg/m²) (54.9%), and normal weight (BMI<25.0 kg/m²) (54.6%) users.

1. (canceled)

2. A method of providing contraception with reduced likelihood of scheduled bleeding in a woman having a BMI≥30.0 kg/m², comprising:
selecting a woman determined to have a BMI≥30.0 kg/m², and then
orally administering to the selected woman a therapeutically effective amount of an estetrol component at a daily dose of from 14 mg to 16 mg, based on the estetrol moiety, and drospirenone at a daily dose of from 2.5 mg to 3.5 mg,
wherein the method comprises administration according to consecutive 28-day cycles of daily administration of the estetrol component and drospirenone on consecutive days 1-24 followed by a hormone-free period without administration of the estetrol component and drospirenone on consecutive days 25-28,
wherein the treated woman has a reduced likelihood of scheduled bleeding on day 25 to day 3 of consecutive 28-day cycles.

3. The method of claim 2, wherein the estetrol component is estetrol monohydrate.

4. The method of claim 2, wherein the estetrol component is estetrol monohydrate and is administered at a daily dose of 15 mg and the drospirenone is administered at a daily dose of 3 mg.

5. The method of claim 2, wherein the method further comprises, prior to the selecting, calculating the BMI of the woman from a measured height and weight of the woman.

6. The method of claim 2, wherein the selecting comprises selecting a woman determined to have a BMI≥30.0 kg/m² and ≤35.0 kg/m².

7. The method of claim 2, wherein the selecting comprises selecting a woman determined to have a BMI≥30.0 kg/m² and determined to be 16-50 years old.

8. The method of claim 2, wherein the selecting comprises selecting a woman determined to have a BMI≥30.0 kg/m² and determined to be 16-25 years old.

9. The method of claim 2, wherein the treated woman has a reduced likelihood of experiencing scheduled bleeding on day 25 to day 3 of a 28-day cycle than a woman having a BMI<30.0 kg/m².

10. (canceled)

11. A method of providing contraception with an increased likelihood of scheduled bleeding in a woman having a BMI<30.0 kg/m², comprising:
selecting a woman determined to have a BMI<30.0 kg/m², and then
orally administering to the selected woman a therapeutically effective amount of an estetrol component at a daily dose of from 14 mg to 16 mg, based on the estetrol moiety, and drospirenone at a daily dose of from 2.5 mg to 3.5 mg,
wherein the method comprises administration according to consecutive 28-day cycles of daily administration of the estetrol component and drospirenone on consecutive days 1-24 followed by a hormone-free period without administration of the estetrol component and drospirenone on consecutive days 25-28,
wherein the woman has an increased likelihood of scheduled bleeding on any of day 25 to day 3 of consecutive 28-day cycles.

12. The method of claim 11, wherein the estetrol component is estetrol monohydrate.

13. The method of claim 11, wherein the estetrol component is estetrol monohydrate and is administered at a daily dose of 15 mg and the drospirenone is administered at a daily dose of 3 mg.

14. The method of claim 11, wherein the method further comprises, prior to the selecting, calculating the BMI of the woman from a measured height and weight of the woman.

15. The method of claim 11, wherein the selecting comprises selecting a woman determined to have a BMI<30.0 kg/m² and determined to be 16-50 years old.

16. The method of claim 11, wherein the selecting comprises selecting a woman determined to have a BMI<30.0 kg/m² and determined to be 16-25 years old.

17. The method of claim 11, wherein the treated woman has an increased likelihood of experiencing scheduled bleeding on day 25 to day 3 of a 28-day cycle than a woman having a BMI≥30.0 kg/m².
